(19) United States
(12) Patent Application Publication
(10) Pub. No.: US 2025/0099126 A1
(43) Pub. Date: Mar. 27, 2025

(54) THROMBECTOMY APPARATUS

(71) Applicant: Boston Scientific Scimed, Inc., Maple Grove, MN (US)

(72) Inventors: Winnie Tang, Santa Clara, CA (US); Roy Leguidleguid, Union City, CA (US); Clifford Van, Santa Clara, CA (US); Dokmai Outama, Stockton, CA (US); Johnathan J. Braun, Brooklyn Park, MN (US); James Burke Lynch, Minneapolis, MN (US)

(73) Assignee: Boston Scientific Scimed, Inc., Maple Grove, MN (US)

(21) Appl. No.: 18/892,652

(22) Filed: Sep. 23, 2024

Related U.S. Application Data
(60) Provisional application No. 63/540,608, filed on Sep. 26, 2023.

(51) Int. Cl. A61B 17/3207; A61B 17/221

(52) U.S. Cl. CPC A61B 17/3207; A61B 2017/2215

(57) ABSTRACT

A thrombectomy apparatus includes a funnel that is secured to a distal end of a catheter. The funnel includes a braided inner layer, a braided outer layer and a reinforcing structure disposed therebetween. The reinforcing structure includes a tubular metal member and a plurality of fingers extending distally from the tubular metal member. Each finger defines two or more pockets. Each of two or more rings extend through a pocket of each of the plurality of fingers, the pockets limiting axial movement of each of the two or more rings. A tractor is adapted to extend over the outer surface of the catheter and the funnel in an un-inverted configuration and to extend in an inverted configuration through the funnel and into the lumen. An elongate member extends through the lumen and is secured to an end of the tractor disposed within the lumen.

### CROSS-REFERENCE TO RELATED APPLICATIONS

This application claims the benefit of priority under 35 U.S.C. § 119 of U.S. Provisional Application No. 63/540,608, filed Sep. 26, 2023, the entire disclosure of which is hereby incorporated by reference.

### TECHNICAL FIELD

The present disclosure relates generally to medical devices. More particularly, the present disclosure pertains to medical devices for performing thrombectomy procedures.

### BACKGROUND

A wide variety of intracorporeal medical devices have been developed for medical use, for example, intravascular use. Some of these devices include thrombectomy apparatuses. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

The disclosure is directed to design, material, manufacturing method, and use alternatives for thrombectomy apparatuses. An example may be found in a thrombectomy apparatus for removing a clot from a vessel. The thrombectomy apparatus includes a catheter having a distal region including a distal end, the catheter defining a lumen extending proximally from the distal end and having an outer surface. A funnel is secured to the distal end and extends distally therefrom. The funnel includes a braided inner layer, a braided outer layer and a reinforcing structure disposed between the braided inner layer and the braided outer layer. The reinforcing structure includes a tubular metal member, a plurality of fingers extending distally from the tubular metal member, each of the plurality of fingers defining two or more pockets, and two or more rings, each of the two or more rings extending through a pocket of each of the plurality of fingers, the pockets limiting axial movement of each of the two or more rings. A tractor is adapted to extend over the outer surface of the catheter and the funnel in an un-inverted configuration and to extend in an inverted configuration through the funnel and into the lumen, the tractor adapted to invert by rolling over the distal end of the funnel when the tractor moves proximally within the lumen. An elongate member extends through the lumen and is secured to an end of the tractor disposed within the lumen.

Alternatively or additionally, the tractor may be adapted to engage the clot and pull the clot proximally into the funnel as the tractor inverts over the distal end of the funnel.

Alternatively or additionally, the tractor may include a flexible tube having a plurality of loops that lie flat before reaching the distal end of the catheter and after passing the distal end of the catheter, but temporarily extend radially outwardly as the tractor inverts over the distal end of the catheter, the plurality of loops temporarily extending radially outward as the tractor inverts over the distal end of the catheter engage the clot and pull the clot proximally.

Alternatively or additionally, each of the plurality of fingers may be doubled over on themselves such that each of the plurality of fingers include an outer finger portion and an inner finger portion.

Alternatively or additionally, the outer finger portion of each of the plurality of fingers may be disposed adjacent the braided outer layer and the inner finger portion of each of the plurality of fingers may be disposed adjacent the braided inner layer.

Alternatively or additionally, each pocket on each of the plurality of fingers may be defined by a first weld proximal of the ring extending through that pocket and a second weld distal of the ring extending through that pocket.

Alternatively or additionally, each first weld and each second weld may extend between the outer finger portion and the inner finger portion.

Alternatively or additionally, the funnel may have a tapered profile tapering from a maximum diameter at a distal end of the funnel to a minimum diameter at a proximal end of the funnel.

Alternatively or additionally, the two or more rings may include a first ring having a first outer diameter, the first ring extending through a first set of pockets of the plurality of pockets, and a second ring having a second outer diameter, the second ring extending through a second set of pockets of the plurality of pockets.

Alternatively or additionally, the two or more rings may further include a third ring having a third outer diameter, the third ring extending through a third set of pockets of the plurality of pockets.

Alternatively or additionally, the braided inner layer may be adapted to allow a guidewire to be advanced through the funnel without snagging on the braided inner layer.

Alternatively or additionally, the thrombectomy apparatus may further include a polymeric sleeve embedded within a proximal region of the braided inner layer.

Another example may be found in a thrombectomy apparatus for removing a clot from a vessel. The thrombectomy apparatus includes a catheter having a distal region including a distal end, the catheter defining a lumen extending proximally from the distal end, the catheter having an outer surface. A funnel is secured to the distal end and extending distally therefrom, the funnel including a braided inner layer and a braided outer layer. A plurality of fingers extend between the braided inner layer and the braided outer layer, each of the plurality of fingers being doubled over on themselves to form an inner finger portion and an outer finger portion. The thrombectomy apparatus includes a plurality of rings, each of the plurality of rings disposed between the inner finger portion and the outer finger portion of each of the plurality of fingers, each of the plurality of rings adapted to limit radial expansion of the funnel. A tractor is adapted to extend over the outer surface of the catheter and the funnel in an un-inverted configuration and to extend in an inverted configuration through the funnel and into the lumen, the tractor adapted to invert by rolling over the distal end of the funnel when the tractor moves proximally within the lumen.

Alternatively or additionally, the thrombectomy apparatus may further include a plurality of pockets formed between the inner finger portion and the outer finger portion of each of the plurality of fingers.

Alternatively or additionally, the plurality of pockets may include a first set of pockets arranged at a first axial position of the funnel to accommodate a first ring of the plurality of rings, the first ring captured within each of the first set of pockets, a second set of pockets arranged at a second axial position of the funnel to accommodate a second ring of the plurality of rings, the second ring captured within each of the second set of pockets, and a third set of pockets arranged at a third axial position of the funnel to accommodate a third ring of the plurality of rings, the third ring captured within each of the third set of pockets.

Alternatively or additionally, each of the plurality of pockets may be defined by a first weld and a second weld each extending between the inner finger portion and the outer finger portion.

Alternatively or additionally, each of the plurality of rings may include a polymeric material.

Alternatively or additionally, at least some of the plurality of rings may include polyester fiber.

Another example may be found in a thrombectomy apparatus for removing a clot from a vessel. The thrombectomy apparatus includes a catheter having a distal region including a distal end, the catheter defining a lumen extending proximally from the distal end, the catheter having an outer surface. A funnel is secured to the distal end and extends distally therefrom. The funnel includes a braided inner layer including a proximal end, a polymeric cuff disposed within the braided inner layer such that the proximal end of the braided inner layer is at least partially embedded within the polymeric cuff, and a braided outer layer. A plurality of fingers extend between the braided inner layer and the braided outer layer, each of the plurality of fingers being doubled over on themselves to form an inner finger portion and an outer finger portion. The thrombectomy apparatus includes a plurality of rings, each of the plurality of rings disposed between the inner finger portion and the outer finger portion of each of the plurality of fingers, each of the plurality of rings adapted to limit radial expansion of the funnel. A tractor is adapted to extend over the outer surface of the catheter and the funnel in an un-inverted configuration and to extend in an inverted configuration through the funnel and into the lumen, the tractor adapted to invert by rolling over the distal end of the funnel when the tractor moves proximally within the lumen.

Alternatively or additionally, the polymeric cuff may be adapted to prevent the braided inner layer from ensnaring a guidewire advanced proximally to distally through the funnel.

The preceding summary is provided to facilitate an understanding of some of the innovative features unique to the present disclosure and is not intended to be a full description. A full appreciation of the disclosure can be gained by taking the entire specification, claims, figures, and abstract as a whole.

### BRIEF DESCRIPTION OF THE DRAWINGS

The disclosure may be more completely understood in consideration of the following description of various examples in connection with the accompanying drawings, in which:

FIG. 1 is a schematic view of an illustrative thrombectomy apparatus shown in a starting position proximate a clot within a blood vessel;

FIG. 2 is a schematic view of the illustrative thrombectomy apparatus of FIG. 1, shown in a finishing position after having captured the clot from within the blood vessel;

FIG. 3 is a schematic view of an illustrative tractor disposed over an illustrative funnel as part of the illustrative thrombectomy apparatus of FIG. 1;

FIG. 4 is a schematic view of a portion of the illustrative thrombectomy apparatus of FIG. 1;

FIG. 5 is a schematic view of an illustrative reinforcing structure forming part of the illustrative thrombectomy apparatus of FIG. 1;

FIG. 6 is a schematic view of an illustrative reinforcing structure forming part of the illustrative thrombectomy apparatus of FIG. 1;

FIGS. 7A and 7B are schematic views of a portion of the illustrative reinforcing structure forming part of the illustrative thrombectomy apparatus of FIG. 1; and

FIG. 8 is a schematic view of an illustrative braided member including a braided outer layer and a braided inner layer, the illustrative braided member forming a funnel that is part of the illustrative thrombectomy apparatus of FIG. 1.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DESCRIPTION

The following description should be read with reference to the drawings, in which like elements in different drawings are numbered in like fashion. The drawings, which are not necessarily to scale, depict examples that are not intended to limit the scope of the disclosure. Although examples are illustrated for the various elements, those skilled in the art will recognize that many of the examples provided have suitable alternatives that may be utilized.

All numbers are herein assumed to be modified by the term “about”, unless the content clearly dictates otherwise. The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is contemplated that the feature, structure, or characteristic may be applied to other embodiments whether or not explicitly described unless clearly stated to the contrary.

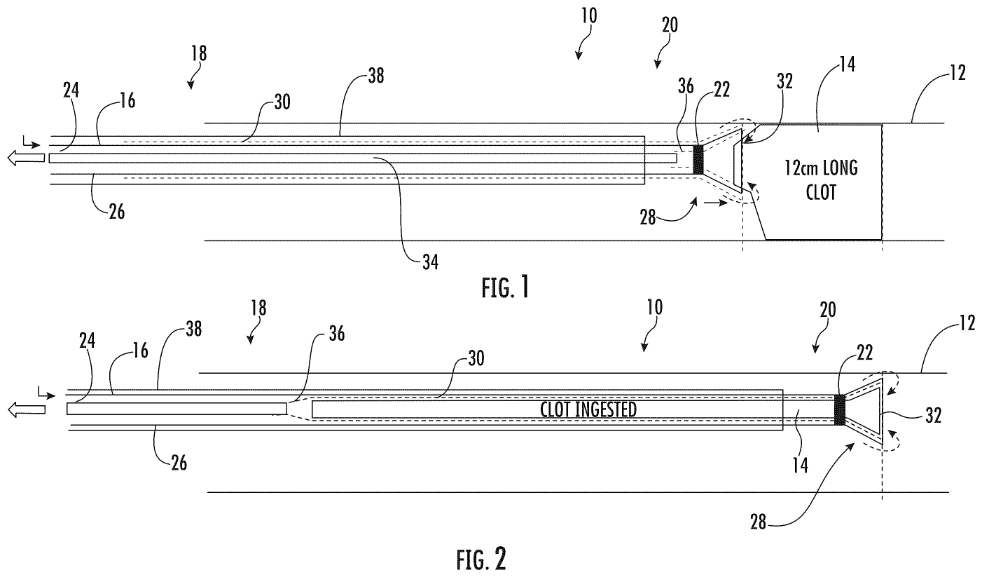

Thrombectomy is a process whereby a thrombus, or clot, is removed from within a blood vessel. FIG. 1 is a schematic view of an illustrative thrombectomy apparatus 10 that is shown positioned within an example blood vessel 12. A thrombus or clot 14 is schematically shown within the blood vessel 12. The thrombectomy apparatus 10 includes a catheter 16 that extends from a proximal region 18 to a distal region 20 and includes a distal end 22. A lumen 24 extends through the catheter 16. The catheter 16 includes an outer surface 26. A funnel 28 is secured to the distal end 22 and extends distally from the distal end 22. In some instances, the funnel 28 is a braided structure including a braided inner layer and a braided outer layer, as will be shown in subsequent drawings. As will be discussed, in some instances the braided outer layer may be adapted to constrain the braided inner layer from expanding too far in a radially outward direction as a result of an axial compressive force.

A tractor 30 is adapted to extend over the outer surface 26 of the catheter 16 and the funnel 28 in an un-inverted configuration and to extend in an inverted configuration through the funnel 28 and into the lumen 24. In some instances, the tractor 30 may be adapted to invert by rolling over a distal end 32 of the funnel 28 when the tractor 30 moves proximally within the lumen 24. The thrombectomy apparatus 10 includes an elongate member 34 that extends through the lumen 24 and is operably coupled with an end 36 of the tractor 30 that is disposed within the lumen 24. In some instances, the tractor 30 is adapted to engage the clot 14 and to pull the clot 14 proximally into the funnel 28 and thus into the lumen 24 as the tractor 30 inverts over the distal end 32 of the funnel 28.

The thrombectomy apparatus 10 includes an elongate member 34 that is adapted to extend through the lumen 24 within the catheter 16. The elongate member 34 is secured to the end 36 of the tractor 30 that is disposed within the lumen 24. In some instances, the elongate member 34 may be a catheter. In some instances, the elongate member 34 may be a pull wire or other pull member that is flexible enough to bend within the lumen 24 as the catheter 16 navigates the vasculature, but strong enough to allow a user to pull the elongate member 34 proximally in order to pull the tractor 30 proximally into the funnel 28 and thus into the lumen 24. In some instances, the elongate member 34 may be adhesively secured to the funnel 28. In some instances, the elongate member 34 may be welded to the funnel 28.

In some instances, the thrombectomy apparatus 10 may include a sheath 38. The sheath 38 may overly the catheter 16 and the tractor 30. In some instances, the sheath 38 helps prevent the tractor 38 from engaging or otherwise contacting the walls of the blood vessel 12. In some instances, the sheath 38 may have an inner diameter sufficient to prevent the tractor 38 from engaging the walls of the blood vessel 12 while still allowing the tractor 30 to move relative to the outer surface 26 of the catheter 16 as the tractor 30 moves. In some instances, the sheath 38 or at least an inner layer or coating may be formed of a lubricious material such as but not limited to a fluoropolymer. As an example, the sheath 38 may be formed of, or may at least have an inner layer or coating that is formed of a fluoropolymer such as polytetrafluoroethylene (PTFE), commonly known as Teflon®.

In some instances, the sheath 38 may be positioned further distally relative to the funnel 28 during advancement of the thrombectomy apparatus 10 through the vasculature. In some instances, the sheath 38 may constrain the funnel 28 into a smaller diameter configuration while the thrombectomy apparatus 10 is advanced through the vasculature. Once a desired location has been reached, the sheath 38 may be withdrawn proximally a short distance in order to allow the funnel 28 to expand into a conical shape, as shown. Prior to subsequent removal of the thrombectomy apparatus 10, the sheath 38 may be advanced distally a short distance in order to once again collapse the funnel 28 into a smaller diameter configuration for navigation.

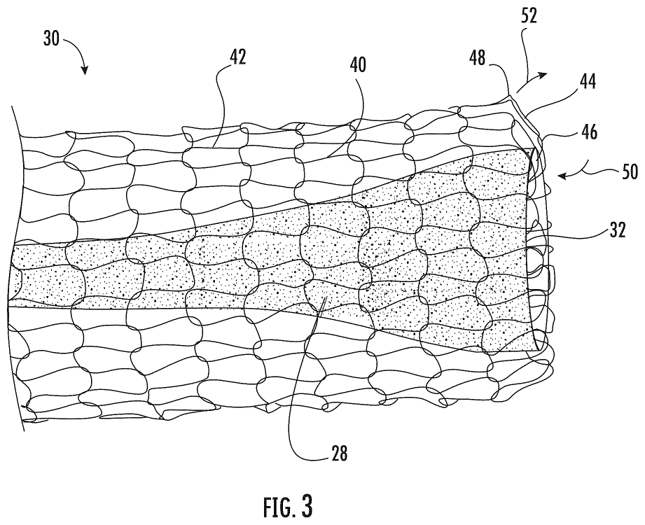

As noted, FIG. 1 shows the thrombectomy apparatus 10 positioned within the blood vessel 12, with the distal end 32 of the funnel 28 positioned adjacent or even in contact with the clot 14. By withdrawing the elongate member 34 proximally, the tractor 30 is caused to move over the distal end 32 of the funnel 28, and to pass through an interior of the funnel 28 and into the lumen 24. In some instances, as the tractor 30 moves from an un-inverted configuration proximal of the distal end 32 of the funnel 28, to an inverted configuration as the tractor 30 moves beyond the distal end 32 of the funnel 28, the tractor 30 is adapted to grab the clot 14 and to pull the clot 14 into the funnel 28 and into the lumen 24. The culmination of this may be seen in FIG. 2, which shows the thrombectomy apparatus 10 in a finishing position in which the clot 14 has been completely pulled into an interior of the thrombectomy apparatus 10. As can be seen in FIG. 2, the clot 14 is positioned within the lumen 24 and within the funnel 28. At this point, withdrawal of the thrombectomy apparatus 10 means that clot 14 will have been removed from the patient.

It will be appreciated that the clot 14 has a length when disposed within the blood vessel 12. In some instances, during the process of capturing the clot 14, the thrombectomy apparatus 10 will move distally. In some instances, the thrombectomy apparatus 10 will move distally a distance about equal to the length of the clot 14 when the clot 14 is still in the blood vessel 12. If the undisturbed clot 14 has a length of 12 millimeters, the thrombectomy apparatus 10 may move distally a distance of about 12 millimeters in the process of capturing the clot 14. In some instances, depending on the nature of the clot 14, the thrombectomy apparatus 10 may move distally a distance greater than the length of the undisturbed clot 14, for example.

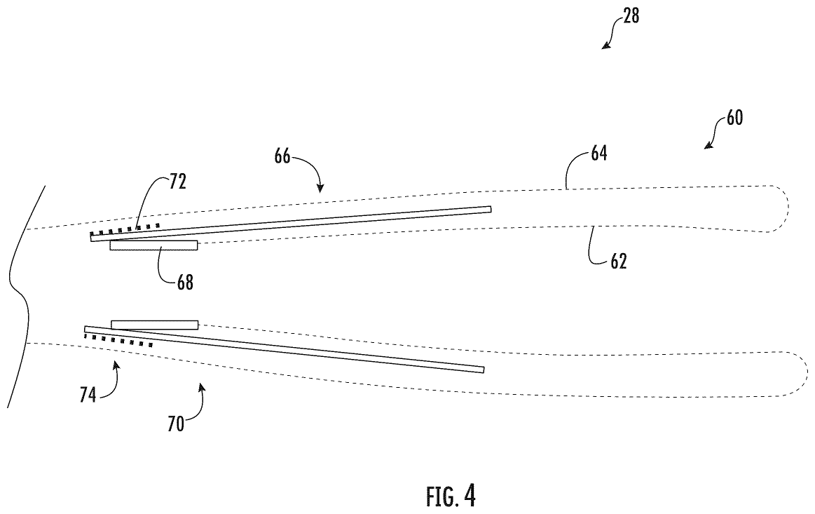

FIG. 3 is a schematic view of the tractor 30 disposed over the funnel 28. In some instances, as shown, the tractor 38 may be formed from a plurality of loops 40. In some instances, the tractor 30 may be formed by knitting one or more filaments 42 into the plurality of loops 40. A variety of different knitting patterns may be utilized, for example. It will be appreciated that the portion of the tractor 30 shown outside of the funnel 28 is in an un-inverted configuration while the portion of the tractor 30 inside the funnel 28 (not visible) is in an inverted configuration. The tractor 30 inverts from the un-inverted configuration to the inverted configuration as the tractor 30 moves around the distal end 32 of the funnel 28. With particular attention to a specific loop 44, it can be seen that the loop 44 has a leading edge 46 and a trailing edge 48. As the loop 44 pivots around the distal end 32 of the funnel 28, the leading edge 46 moves in a direction indicated by an arrow 50 while the trailing edge 46 moves in a direction indicated by an arrow 52. Accordingly, as each of the loops 44 pivot around the distal end 32 of the funnel 28, each loop 44 will temporarily extend radially outwardly as the loop 44 pivots. Otherwise, each of the loops 44 will lay flat, particularly as tension is placed on the tractor 30. Additional details regarding the thrombectomy apparatus 10, including the catheter 16, the tractor 30 and the elongate member 34, may be found in U.S. Pat. Nos. 9,962,178; 10,835,269; US 2022/0257269 and US 2022/0287053, each of which are incorporated by reference herein. It will be appreciated that as the tractor 30 is pulled into the funnel 28 and into the lumen 24 by withdrawing the elongate member 34 proximally, and as a result the tractor 30 inverts over the distal end 32 of the funnel 28, that an axial compressive force is applied to the funnel 28 by the tractor 30. In some instances, the funnel 28 may be adapted to resist or otherwise accommodate this axial compressive force such that the funnel 28 is able to retain its tapered shape. In some instances, as will be discussed, the funnel 28 may be a braided structure. Braided structures can have a tendency to increase in diameter in response to a decrease in length, and of course the inverse is true as well. A braided structure can have a tendency to decrease in diameter in response to an increase in length. In some instances, the funnel 28 may be a braided structure having an inner braided layer and an outer braided layer.

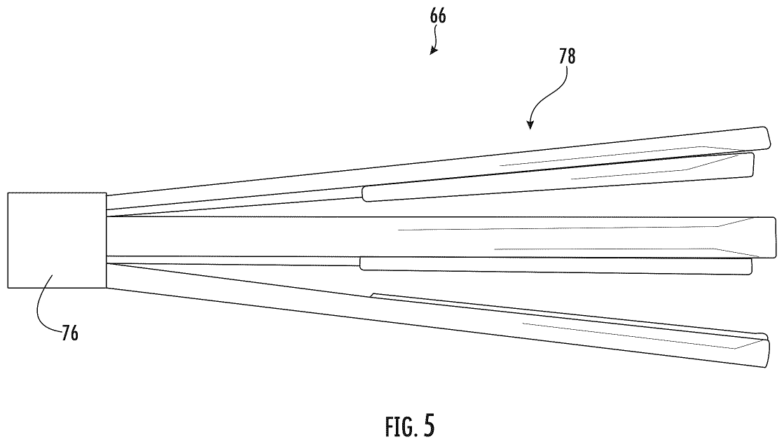

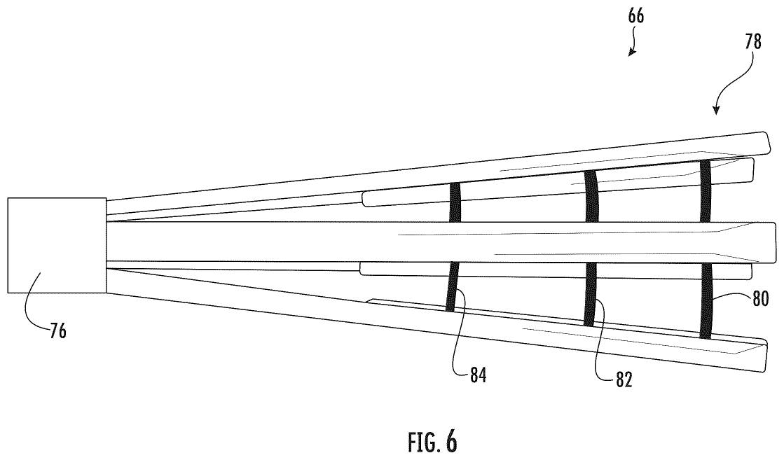

In some instances, the thrombectomy apparatus 10 may include a reinforcing structure disposed within or otherwise forming part of the funnel 28. The reinforcing structure may be adapted to help the funnel 28 retain a conical shape even when the funnel 28 is undergoing an axial compressive force as a result of the tractor 30 being pulled proximally over the distal end 32 of the funnel 28. FIG. 4 is a schematic view showing several features of the funnel 28. The funnel 28 includes a braided member 60 that is folded over on itself to have a braided inner layer 62 and a braided outer layer 64. A reinforcing structure 66 may be seen as being disposed between the braided inner layer 62 and the braided outer layer 64. The reinforcing structure 66 may take multiple forms, as will be shown for example in FIGS. 5, 6, 7A and 7B. In some instances, as shown, the funnel 28 may be considered as having a tapered profile tapering from a maximum diameter at the distal end 32 of the funnel 28 to a minimal diameter at an opposing proximal end.

A polymeric braid cuff 68 is disposed within the braided inner layer 62. In some instances, the polymeric braid cuff 68 is embedded within a proximal end region 70 of the braided inner layer 62. In some instances, the polymeric braid cuff 68 may at least partially embed any lose ends of the filament or filaments forming the braided member 60 such that an interior of the braided inner layer 62 forms a smooth surface that is free or at least substantially free of any loose filament ends that could otherwise interfere with being able to subsequently extend a guidewire distally through the thrombectomy apparatus 10 after having advanced the thrombectomy apparatus 10 over a guidewire that was removed after positioning the thrombectomy apparatus 10.

The polymeric braid cuff 68 may be formed of any desired polymeric material, including those materials listed below. In some instances, the polymeric braid cuff 68 may be formed of 68D Pebax®, but this is just an example. In some instances, the polymeric braid cuff 68 may be secured in position within the proximal end region 70 of the braided inner layer 62 via a thermal process. The polymeric braid 68 may be placed on a mandrel, followed by positioning the braided inner layer 62 with its proximal end region 70 partially overlapping with the polymeric braid cuff 68. A heat shrink tube may be positioned over the polymeric braid cuff 68 and the proximal end region 70 of the braided inner layer 62. Heat may be applied to shrink the heat shrink tube and thus compress the proximal end region 70 of the braided inner layer 62 into the polymeric braid cuff 68. The heat shrink tube may subsequently be cut off prior to further manufacturing steps. In some instances, the thrombectomy apparatus 10 may also include a PET ring 72 that overlays a proximal region 74 of the reinforcing structure 66.

FIG. 5 is a schematic view of the reinforcing structure 66. The reinforcing structure 66 includes a tubular base member 76 and a plurality of fingers 78 extending distally from the tubular base member 76. In some instances, for example, the reinforcing structure 66 may be formed by cutting longitudinal slits into a hypotube to form a number of finger blanks. The longitudinal slits may be laser-cut, for example. Each of the finger blanks can be folded back over itself, and each of the finger blanks may be splayed somewhat to form the plurality of fingers 78. In some instances, the reinforcing structure 66 may further include a number of fiber force rings that help to limit radial movement of the fingers 78.

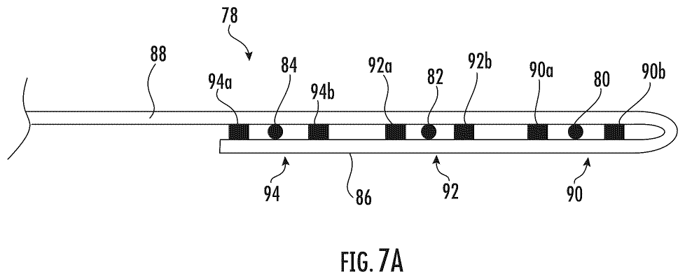

FIG. 6 shows the reinforcing structure 66 with the addition of a first ring 80, a second ring 82 and a third ring 84. As noted, each of the fingers 78 are bent over itself, forming a space between an inner finger portion and an outer finger portion. The rings 80, 82 and 84 may extend between the inner finger portion and the outer finger portion of each of the fingers 78. While a total of three rings 80, 82 and 84 are shown, it will be appreciated that this is merely illustrative. In some instances, the reinforcing structure 66 may include only one or two rings. In some instances, the reinforcing structure 66 may include four or more rings. The rings 80, 82 and 84 may be formed of a polymeric material. In some instances, the rings 80, 82 and 84 may be formed of polyester fiber.

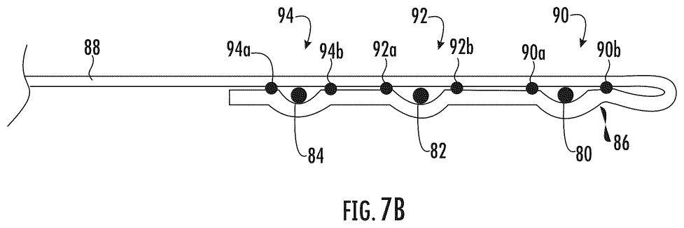

In some instances, the rings 80, 82 and 84 may be limited in axial travel by extending through pockets formed within each of the fingers 78. FIG. 7A is a schematic view of a single finger 78, showing an inner finger portion 86 and an outer finger portion 88. When the funnel 28 is assembled, the inner finger portion 86 of each of the fingers 78 may be disposed adjacent the braided inner layer 62 and the outer finger portion 88 of each of the fingers 78 may be disposed adjacent the braided outer layer 64. The ring 80 may be seen as extending through a pocket 90, the ring 82 may be seen as extending through a pocket 92 and the ring 84 may be seen as extending through a pocket 94. The pocket 90 may be formed by forming a first weld 90a on a first side of the pocket 90 and a second weld 90b on a second side of the pocket 90, with the ring 80 thus captured within the pocket 90. As can be seen, the pocket 90 will constrain axial movement of the ring 80. Similarly, the pocket 92 may be formed by forming a first weld 92a on a first side of the pocket 92 and a second weld 92b on a second side of the pocket 92, with the ring 82 thus captured within the pocket 92. As can be seen, the pocket 92 will constrain axial movement of the ring 82. The pocket 94 may be formed by forming a first weld 94a on a first side of the pocket 94 and a second weld 94b on a second side of the pocket 94, with the ring 84 thus captured within the pocket 94. As can be seen, the pocket 94 will constrain axial movement of the ring 84.

In some instances, the first ring 80 may extend through a first set of pockets 90 formed in each of the plurality of fingers 78. The second ring 82 may extend through a second set of pockets 92 formed in each of the plurality of fingers 78. The third ring 84 may extend through a third set of pockets 94 formed in each of the plurality of fingers 78. During assembly, the rings 80, 82 and 84 may be placed in position extending between the inner finger portion 86 and the outer finger portion 88 prior to forming the welds 90a, 90b, 92a, 92b, 94a and 94b. In some instances, each of the welds 90a, 90b, 92a, 92b, 94a and 94b may be considered as extending between the inner finger portion 86 and the outer finger portion 88. In some instances, as shown in FIG. 7A, a gap may remain between the inner finger portion 86 and the outer finger portion 88 of each of the fingers 78. FIG. 7A is schematic in nature, as the inner finger portion 86 may not remain linear, but may be pressed towards the outer finger portion 86 during the manufacturing process. In some instances, as shown for example in FIG. 7B, the inner finger portion 86 may come into contact with the outer finger portion 86 in regions between adjacent pockets 90, 92, 94, for example.

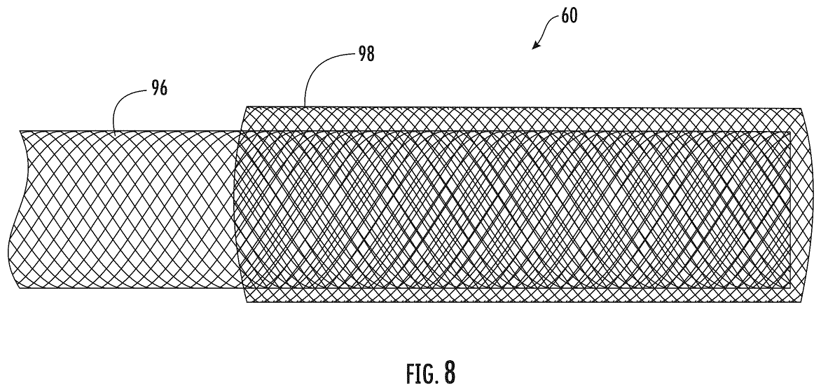

FIG. 8 is a schematic view of the braided member 60 before being constrained to form the funnel 28. As can be seen, the braided member 60 includes a first portion 96 that will form the braided inner layer 62 and a second portion 98 that will form the braided outer layer 64. The second portion 98 has been doubled over the first portion 96. During the manufacturing process, first the braided inner layer 62 and then the braided outer layer 64 will be constrained into the conical shape of the funnel 28. In some instances, the first portion 96 and the second portion 98 of the braided member 60 may be formed by braiding the braided member 60 from one or more filaments, with an similar braid angle throughout the braided member 60. It will be appreciated that the braid angle will change considerably when the braided member 60 is compressed down into the funnel 28.

The materials that can be used for the various components of the medical stent(s), and the various elements thereof disclosed herein may include those commonly associated with medical devices. For simplicity purposes, the following discussion refers to the apparatus. However, this is not intended to limit the devices and methods described herein, as the discussion may be applied to other elements, members, components, or devices disclosed herein, such as, but not limited to, the medical stent and/or elements or components thereof. In some instances, the apparatus, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-NR and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some instances, portions or all of the apparatus, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the apparatus in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the apparatus to achieve the same result.

In some instances, a degree of Magnetic Resonance Imaging (MRI) compatibility is imparted into the apparatus and/or other elements disclosed herein. For example, the apparatus, and/or components or portions thereof, may be made of a material that does not substantially distort the image and create substantial artifacts (e.g., gaps in the image). Certain ferromagnetic materials, for example, may not be suitable because they may create artifacts in an MRI image. The apparatus, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some instances, the apparatus and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

Having thus described several illustrative examples of the present disclosure, those of skill in the art will readily appreciate that yet other examples may be made and used within the scope of the claims hereto attached. It will be understood, however, that this disclosure is, in many respects, only illustrative. Changes may be made in details, particularly in matters of shape, size, arrangement of parts, and exclusion and order of steps, without exceeding the scope of the disclosure. The disclosure's scope is, of course, defined in the language in which the appended claims are expressed.

What is claimed is:

1. A thrombectomy apparatus for removing a clot from a vessel, the thrombectomy apparatus comprising:
a catheter having a distal region including a distal end, the catheter defining a lumen extending proximally from the distal end, the catheter having an outer surface;
a funnel secured to the distal end and extending distally therefrom, the funnel including:
a braided inner layer; and
a braided outer layer;
a reinforcing structure disposed between the braided inner layer and the braided outer layer, the reinforcing structure comprising:
a tubular base member;
a plurality of fingers extending distally from the tubular base member, each of the plurality of fingers defining two or more pockets; and
two or more rings, each of the two or more rings extending through a pocket of each of the plurality of fingers, the pockets limiting axial movement of each of the two or more rings;
a tractor adapted to extend over the outer surface of the catheter and the funnel in an un-inverted configuration and to extend in an inverted configuration through the funnel and into the lumen, the tractor adapted to invert by rolling over the distal end of the funnel when the tractor moves proximally within the lumen; and
an elongate member extending through the lumen and secured to an end of the tractor disposed within the lumen.

2. The thrombectomy apparatus of claim 1, wherein the tractor is adapted to engage the clot and pull the clot proximally into the funnel as the tractor inverts over the distal end of the funnel.

3. The thrombectomy apparatus of claim 2, wherein the tractor comprises a flexible tube comprising a plurality of loops that lie flat before reaching the distal end of the catheter and after passing the distal end of the catheter, but temporarily extend radially outwardly as the tractor inverts over the distal end of the catheter, the plurality of loops temporarily extending radially outward as the tractor inverts over the distal end of the catheter engage the clot and pull the clot proximally.

4. The thrombectomy apparatus of claim 1, wherein each of the plurality of fingers are doubled over on themselves such that each of the plurality of fingers include an outer finger portion and an inner finger portion.

5. The thrombectomy apparatus of claim 4, wherein the outer finger portion of each of the plurality of fingers is disposed adjacent the braided outer layer and the inner finger portion of each of the plurality of fingers is disposed adjacent the braided inner layer.

6. The thrombectomy apparatus of claim 4, wherein each pocket on each of the plurality of fingers is defined by a first weld proximal of the ring extending through that pocket and a second weld distal of the ring extending through that pocket.

7. The thrombectomy apparatus of claim 6, wherein each first weld and each second weld extend between the outer finger portion and the inner finger portion.

8. The thrombectomy apparatus of claim 1, wherein the funnel has a tapered profile tapering from a maximum diameter at a distal end of the funnel to a minimum diameter at a proximal end of the funnel.

9. The thrombectomy apparatus of claim 1, wherein the two or more rings comprise:
a first ring having a first outer diameter, the first ring extending through a first set of pockets of the plurality of pockets; and
a second ring having a second outer diameter, the second ring extending through a second set of pockets of the plurality of pockets.

10. The thrombectomy apparatus of claim 9, wherein the two or more rings further comprise:
a third ring having a third outer diameter, the third ring extending through a third set of pockets of the plurality of pockets.

11. The thrombectomy apparatus of claim 1, wherein the braided inner layer is adapted to allow a guidewire to be advanced through the funnel without snagging on the braided inner layer.

12. The thrombectomy apparatus of claim 11, further comprising a polymeric braid cuff embedded within a proximal region of the braided inner layer.

13. A thrombectomy apparatus for removing a clot from a vessel, the thrombectomy apparatus comprising:
a catheter having a distal region including a distal end, the catheter defining a lumen extending proximally from the distal end, the catheter having an outer surface;
a funnel secured to the distal end and extending distally therefrom, the funnel including:
a braided inner layer; and
a braided outer layer;
a plurality of fingers extending between the braided inner layer and the braided outer layer, each of the plurality of fingers being doubled over on themselves to form an inner finger portion and an outer finger portion; and
a plurality of rings, each of the plurality of rings disposed between the inner finger portion and the outer finger portion of each of the plurality of fingers, each of the plurality of rings adapted to limit radial expansion of the funnel; and
a tractor adapted to extend over the outer surface of the catheter and the funnel in an un-inverted configuration and to extend in an inverted configuration through the funnel and into the lumen, the tractor adapted to invert by rolling over the distal end of the funnel when the tractor moves proximally within the lumen.

14. The thrombectomy apparatus of claim 13, further comprising a plurality of pockets formed between the inner finger portion and the outer finger portion of each of the plurality of fingers.

15. The thrombectomy apparatus of claim 14, wherein the plurality of pockets comprise:
a first set of pockets arranged at a first axial position of the funnel to accommodate a first ring of the plurality of rings, the first ring captured within each of the first set of pockets;
a second set of pockets arranged at a second axial position of the funnel to accommodate a second ring of the plurality of rings, the second ring captured within each of the second set of pockets; and
a third set of pockets arranged at a third axial position of the funnel to accommodate a third ring of the plurality of rings, the third ring captured within each of the third set of pockets.

16. The thrombectomy apparatus of claim 14, wherein each of the plurality of pockets are defined by a first weld and a second weld each extending between the inner finger portion and the outer finger portion.

17. The thrombectomy apparatus of claim 13, wherein each of the plurality of rings comprise a polymeric material.

18. The thrombectomy apparatus of claim 13, wherein at least some of the plurality of rings comprise polyester fiber.

19. A thrombectomy apparatus for removing a clot from a vessel, the thrombectomy apparatus comprising:
a catheter having a distal region including a distal end, the catheter defining a lumen extending proximally from the distal end, the catheter having an outer surface;
a funnel secured to the distal end and extending distally therefrom, the funnel including:
a braided inner layer including a proximal end;
a polymeric braid cuff disposed within the braided inner layer such that the proximal end of the braided inner layer is at least partially embedded within the polymeric cuff; and
a braided outer layer;
a plurality of fingers extending between the braided inner layer and the braided outer layer, each of the plurality of fingers being doubled over on themselves to form an inner finger portion and an outer finger portion; and
a plurality of rings, each of the plurality of rings disposed between the inner finger portion and the outer finger portion of each of the plurality of fingers, each of the plurality of rings adapted to limit radial expansion of the funnel; and
a tractor adapted to extend over the outer surface of the catheter and the funnel in an un-inverted configuration and to extend in an inverted configuration through the funnel and into the lumen, the tractor adapted to invert by rolling over the distal end of the funnel when the tractor moves proximally within the lumen.

20. The thrombectomy apparatus of claim 19, wherein the polymeric cuff is adapted to prevent the braided inner layer from ensnaring a guidewire advanced proximally to distally through the funnel.
